(19) United States
(12) Patent Application Publication
(10) Pub. No.: US 2013/0172968 A1
(43) Pub. Date: Jul. 4, 2013

(54) Therapuedic undergarments/garments for treatment of Fibromyalgia (FM)

(71) Applicant: Cathryn Darlene Turley, Beverly Hills, FL (US)

(72) Inventor: Cathryn Darlene Turley, Beverly Hills, FL (US)

(21) Appl. No.: 13/342,295

(22) Filed: Jan. 3, 2012

(51) Int. Cl. A61F 7/08

(52) U.S. Cl. 607/112

(57) ABSTRACT

Disclosed is a therapeutic garment for treating patients with muscoskeletal syndromes characterized by pain and/or aversion to changes in weather and hypoxia of the tender points in Fibromyalgia Patients and other Rheumatoid conditions as in Fibromyalgia Syndrome (FM)

### BACKGROUND

Fibromyalgia (FM) is a chronic disorder characterized by widespread musculoskeletal pain, fatigue, and tenderness in localized areas of the neck, spine, shoulders, and hips called “tender points.”

People with this syndrome may also experience sleep disturbances, morning stiffness, irritable bowel syndrome, anxiety, and other symptoms. Available data suggest that the number of persons aged 18 and older in the United States with Fibromyalgia is approximately 3.7 million. It primarily occurs in women of childbearing age, but children, the elderly, and men may also be affected.

Fibromyalgia may be associated with changes in muscle metabolism, such as decreased blood flow, causing fatigue and decreased strength. The American College of Rheumatology (ACR) has developed criteria for Fibromyalgia that physicians can use in diagnosing the disease. According to ACR criteria, a person is considered to have Fibromyalgia if he or she has widespread pain for at least 3 months in combination with tenderness in at least 11 of 18 specific tender point sites

### SUMMARY

The present invention provides an effective method to reduce or eliminate hypoxia on the skin over the tender points and increase circulation and oxygen levels Fibromyalgia (FM) Patients and any other therapeutic applications where warming the tender points/joints/spine eases pain.

### BRIEF DESCRIPTION OF DRAWING

This drawing shows the front and back areas of the upper and lower garment to be heated to cover the Tender Points and Joints for Fibromyalgia (FM) and any other therapeutic uses.

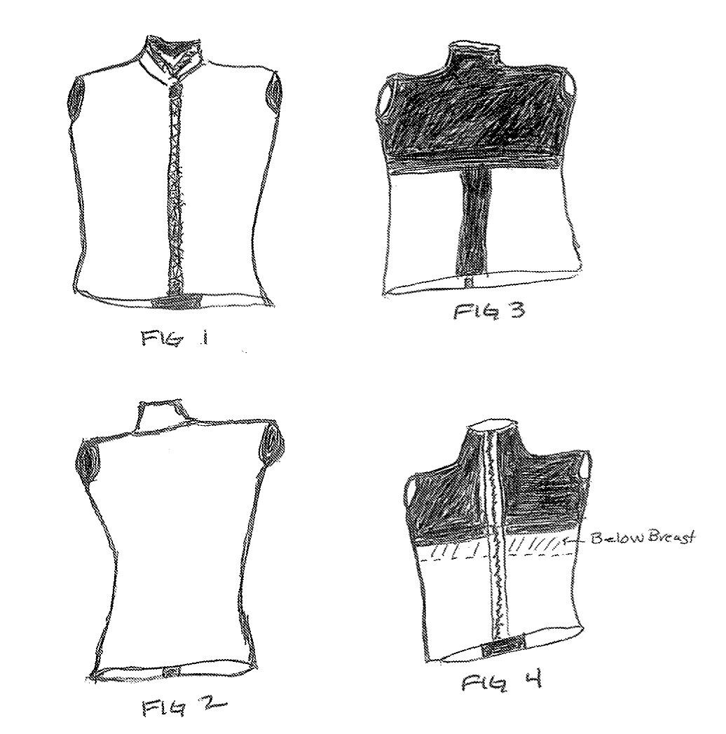

Page 1 FIG. 1 Exterior Frontal Zippered neck to chin view of upper garment w/o sleeves

Page 1 FIG. 2 Exterior Back view of upper garment

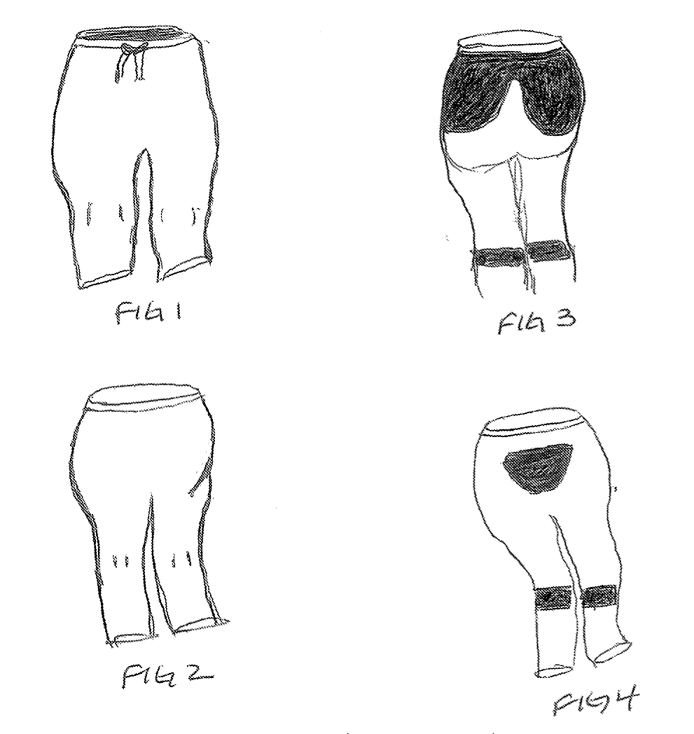

Page 1 FIG. 3 Interior Back view of upper garment (shaded area showing warming material)

Page 1 FIG. 4 Interior Frontal view of upper garment (Shaded area is warming material)

Page 2 FIG. 1 Exterior Frontal view of lower garment with drawstring or elastic waist/

Page 2 FIG. 2 Exterior Rear view of lower garment

Page 2 FIG. 3 Interior Rear view of lower garment (Shaded area showing warming material)

Page 2 FIG. 4 Interior Frontal view of lower garment (Shaded area showing warming material)

Sleeves would consist of Exterior Fabric and warming material around the elbow to warm joint area.

### DETAILED DESCRIPTION OF INVENTION

My invention is a zippered upper and lower undergarment or garment utilizing comfortable exterior and warming Fabrics. Cotton, Muslin, etc. For exterior fabric Fleece, Bio Ceramic, Far Infrared Ray Fabric and or Nano technologies covering the heated areas and another soft, breathable fabric for the non-heated areas.

The Tender Points to be treated are located:

On the anterior upper and lower neck and the upper chest just below the anterior left & right collar bone. Option for warming material to cover breast for sufferers who cannot tolerate to wear a Bra.

Abdomen warmth option provided for IBS sufferers which accompanies Fibromyalgia

On the posterior of the upper body, the posterior of the neck, posterior upper and lower Shoulders, Shoulder Blades and down and aside the Spinal Column. The Posterior Lower Body would cover the lower spine and moving around the Lower Back around towards the Anterior of the Hips.

Sleeves, Lower Legs, Breast and Abdomen coverage is optional upon request.

What is claimed is:

1. A method of comfortable continued warmth treatment of Fibromyalgia Syndrome and Non Inflammatory Rheumatoid Conditions manifested in a human patient with Muscoskeletal or Joint Pain reactive to temperature changes reaching these Hypoxic areas where warmth increases Circulation bringing Oxygen to the Tender Point and Joints reducing or eliminating pain/fiber flares)

2. The method of claim 1 where said syndrome is Fibromyalgia Syndrome (FM)
